(19) United States
(12) Patent Application Publication
(10) Pub. No.: US 2019/0367984 A1
(43) Pub. Date: Dec. 5, 2019

(54) METHODS FOR PREDICTING RESPONSE TO ANTI-TNF THERAPY

(71) Applicant: THE UNIVERSITY OF LIVERPOOL, Liverpool (GB)

(72) Inventors: Helen Louise WRIGHT, Liverpool, Merseyside (GB); Robert John MOOTS, Liverpool, Merseyside (GB); Steven William EDWARDS, Liverpool, Merseyside (GB)

(21) Appl. No.: 15/780,762

(22) PCT Filed: Dec. 2, 2016
(86) PCT No.: PCT/GB2016/053798

§ 371 Date: Jun. 1, 2018

(30) Foreign Application Priority Data
Dec. 3, 2015 (GB) 1521357.2

(51) Int. Cl. C12Q 1/6883; G16H 50/30; G16B 40/10

(52) U.S. Cl. CPC C12Q 1/6883; G16H 50/30; C12Q 2600/136; C12Q 2600/106; C12Q 2600/158; G16B 40/10

(57) ABSTRACT

The present invention relates to a method for predicting the response of a patient suffering from an autoimmune or immune-mediated disorder to anti-TNF therapy based upon the expression of a Low Density Granulocyte gene or one or more interferon regulated biomarkers. Also provided is a kit for performing the invention, and related methods of treatment and monitoring response to treatment.

The present invention relates to a method for predicting the response of a patient suffering from an autoimmune or immune-mediated disorder to anti-TNF therapy.

### INCORPORATION BY REFERENCE OF SEQUENCE LISTING

The Sequence Listing in the ASCII text file, named as 32028_SEQ.txt32028_SEQ_2.txt of 73 KB, created on Feb. 12, 2019, and submitted to the United States Patent and Trademark Office via EFS-Web, is incorporated herein by reference.

### BACKGROUND

Rheumatoid arthritis (RA) is a systemic inflammatory disorder which causes disability and poor quality of life for over 500,000 adults in the UK. It also causes premature mortality. Rheumatoid arthritis attacks the synovial fluid of a joint, resulting in inflammation and thickening of the joint capsule. The affected joints become tender, warm and swollen, and movement becomes restricted due to stiffening of the joint. The most commonly affected joints are those of the hands, feet and cervical spine, but larger joints such as the shoulder and knee can also be affected. Many other organs can also be affected by this condition, such as eyes, heart, lungs and skin Rheumatoid arthritis is currently believed to be the result of a combination of genetic and environmental factors.

The disease is heterogeneous and response to drug therapy varies widely between affected individuals. The 2010 American College of Rheumatology (ACR) and the European League Against Rheumatism (EULAR) Rheumatoid Arthritis Classification Criteria were introduced in order to be able to better identify those who were likely to develop a chronic condition. The classification criteria establish a point value between 0 and 10 based upon criteria including joint involvement, serological parameters including rheumatoid factor (RF) and ACPA (Anti-Citrullinated Protein Antibody), acute phase reactants, and duration of arthritis.

A score of 6 or greater unequivocally classifies a person with a diagnosis of rheumatoid arthritis. Serology and autoimmune diagnostics are given major weight in order to better diagnose the condition early before joint destructions occurs.

There are many tools available for monitoring remission in rheumatoid arthritis. Disease Activity Score of 28 joints (DAS28) is widely used as an indicator of RA disease activity and response of the subject to treatment. Using a DAS28 score, the disease activity of the affected person can be classified.

Treatment of rheumatoid arthritis aims to minimize pain and swelling, prevent bone deformity, and maintain day-to-day functioning. Frontline therapies for Rheumatoid arthritis include disease-modifying anti-rheumatic drugs (DMARDs) such as methotrexate. Whilst many patients respond well to DMARDs, a significant proportion (around 30%) fail to achieve adequate disease control.

Tumour Necrosis Factor (TNF) is involved in clinical problems associated with autoimmune and immune-mediated disorders such as rheumatoid arthritis, ankylosing spondylitis, inflammatory bowel disease, psoriasis, psoriatic arthritis hidradenitis suppurativa and refractory asthma. Patients who do not respond to DMARDs typically may be prescribed biologic-DMARDs, such as anti-TNF. Biologics are expensive and so are only available in the UK to patients with the highest level of disease activity (DAS28>5.1). Similarly, patients with other inflammatory arthritides such as psoriatic arthritis and ankylosing spondylitis are required to demonstrate failure to respond to first line medicines and high disease activity to qualify for therapy with an anti-TNF therapy. Unfortunately around 40% of patients receiving anti-TNF therapy fail to achieve or maintain an adequate response, and a trial and error approach has to be taken with a series of alternative biologic-DMARDs until a suitable therapy can be found. This prolonged delay in achieving adequate disease control in drug-resistant patients causes severe and irreversible damage to their joints and wastes valuable healthcare resources. For example, a year's treatment with anti-TNF therapy currently costs around £12,000 per patient therefore placing considerable burden on the healthcare and welfare systems.

For these reasons, it has become desirable to be able to predict a patient's likely response to anti-TNF therapy.

Sekiguchi et al (Rheumatology 2008 47:780-788) describes the identification of a set of genes including OAS1, OAS2 and IFIT1 whose expression differs between responders and non-responders to the anti-TNF biologic, infliximab, in the treatment of rheumatoid arthritis.

WO2008/132176 describes a method for evaluating the response of a patient to anti-TNF therapy for treating rheumatoid arthritis, using increased expression of biomarkers including IFI44 and LY6E to categorise a patient as a good responder. The assay is conducted on synovial fluid from the patient.

US2009/0142769 describes the identification of patients having a disease such as rheumatoid arthritis who will respond to anti-TNF therapy, by detecting the expression of at least one interferon-inducible gene, selected from CXCL10, C1orf29. MX1, IFIT1, IFI44, PRKR, OAS3, GBP1, IRF1, SERPING1, CXC, CXCL9, CXCI10, PSMB8, GPR105, CD64, FCGR1A, IL-1ra, TNRSF1B. The authors also show that a higher IFNβ/α ratio is indicative of a good response to anti-TNF therapy.

WO2012/066536 describes the identification of responders or non-responders to anti-TNF therapy. The biomarkers include expression of IFIT1 and IFI44 as indicating good response.

Identification of responders to anti-TNF therapy is useful, but such methods may identify those patients who are good responders to anti-TNF therapy. It can be seen that it improvements are needed in being able to better identify those patients who are not likely to respond to anti-TNF therapy.

### SUMMARY OF THE INVENTION

In a first aspect, there is provided a method for predicting the response of a subject having an autoimmune or immune-mediated disorder to anti-TNF therapy, wherein the method comprises analysing a sample obtained from the subject to determine the level of a target molecule indicative of the expression of a Low Density Granulocyte (LDG) gene, wherein an elevated level of the target molecule compared to a reference value predicts a non-favourable response of the subject to anti-TNF therapy.

An LDG gene may be a gene specifically expressed by an LDG cell. It may be one or more genes selected from the group consisting of: AZU1, BPI, CEACAM8, CRISP3, CTSG, DEFA4, ELANE, LCN2, LTF, MMP8, MPO, RNASE2, RNASE3.

In a second aspect, the present invention provides a method for predicting the response of a subject having an autoimmune or immune-mediated disorder to anti-TNF therapy, wherein the method comprises analysing a sample obtained from the subject to determine the level of a target molecule indicative of the expression one or more interferon regulated biomarkers selected from the group consisting of: CMPK2, IFI6, RSAD2, and USP18, wherein an elevated level of the target molecule compared to a reference value predicts a favourable response of the subject to anti-TNF therapy.

In an embodiment, the second aspect may further comprise determining the level of a target molecule indicative of the expression one or more interferon regulated biomarkers selected from the group consisting of IFFI44L LY6E, OAS1, OAS2, OAS3 and IFIT1B.

In a third aspect, the present invention provides a method for predicting the response of a subject having an autoimmune or immune-mediated disorder to anti-TNF therapy, wherein the method comprises analysing a sample obtained from the subject to determine the level of i) a target molecule indicative of the expression of a Low Density Granulocyte (LDG) gene and ii) a target molecule indicative of the expression one or more interferon regulated biomarkers selected from the group consisting of: CMPK2, IFI6, RSAD2, USP18, IFFI44L LY6E, OAS1, OAS2, OAS3 and IFIT1B; wherein no substantial elevation in the level of i) and an elevation in the level of ii) compared to a reference value predicts a favourable response of the subject to anti-TNF therapy.

### BRIEF DESCRIPTION OF THE DRAWINGS

Embodiments of the invention are further described hereinafter with reference to the accompanying drawings, in which:

FIG. 1 shows the results of Ingenuity (IPA) analysis. IPA predicted that (A) Interferon Signalling was significantly up-regulated in TNFi responders prior to commencement of therapy, and that (B) CSF3 (G-CSF) regulated genes were down-regulated in TNFi responders and therefore conversely upregulated in TNFi non-responders, where *=upregulated, §=down-regulated, grey=no change.

FIG. 2 is a graphical representation of the study performed on peripheral blood neutrophils from rheumatoid arthritis patients. Expression levels (Reads per Kilobase of Transcript per Million map reads (RPKM)) of the 10 IFN-related genes and 13 LDG-genes which were identified as significantly differentially expressed between TNFi responders and non-responders (edgeR FDR<0.05) in the Original Cohort. Response is measured as the decrease in DAS28 from week 0 to week 12. A decrease in DAS28 of 1.2 or greater is classed as a response.

FIG. 3 is a graphical representation of expression levels of RPKM of the original cohort. Expression levels (RPKM) of the 10 IFN-related genes and 13 LDG-genes in TNFi “Good” responders and non-responders from the Original Cohort. Response is measured as the decrease in DAS28 from week 0 to week 12 using EULAR criteria for Good and Non-Response.

FIG. 4 is a graphical representation of the validation study. Expression levels (qPCR MNE) of the 10 IFN-related genes and 13 LDG-genes in TNFi “Good” responders and non-responders from the Validation Cohort. Response is measured as the decrease in DAS28 from week 0 to week 12 using EULAR criteria for Good and Non-Response.

FIG. 5 is a graphical representation of the expression levels in DMARD naïve patients in the validation cohort. Expression levels (qPCR MNE) of the 10 IFN-related genes and 13 LDG-genes in DMARD-naïve patients from the Validation Cohort. Response is measured as the decrease in DAS28 from week 0 to week 12 using EULAR criteria for Good and Non-Response to DMARDs.

FIG. 6 shows a stepwise regression analysis of 10 IFN-regulated and 13 LDG-genes to identify a good subset of predictor genes.

### DETAILED DESCRIPTION OF THE INVENTION

The present invention is based upon the identification and validation of a gene expression profile which predicts those subjects who will not respond to anti-TNF therapy. Specifically, using transcriptome profiling (RNA-Seq) of peripheral blood neutrophils, expression of a panel of Low Density Granulocyte (LDG) genes has been correlated with response to anti-TNF treatment in subjects having an autoimmune or immune-mediated disorder such as rheumatoid arthritis. The present invention is additionally based upon the identification of a further expression profile of interferon related genes which correlate with good response to anti-TNF response. These two gene expression profiles are mutually exclusive and therefore provide a high degree of sensitivity and specificity for the prediction of response to anti-TNF therapy in an autoimmune or immune-mediated disorder.

The present invention therefore provides the possibility of a clinical test to predict response to anti-TNF therapy, preferably prior to a subject commencing anti-TNF therapy. Such a test will inform the clinician whether the patient is likely to respond to anti-TNF therapy or not, and enable the clinician to commence alternative therapy if the patient is predicted to be unlikely to respond. This will benefit the patient by targeting their treatment with an appropriate therapy early, rather than relying on the current “trial and error” approach. Such a test will therefore enable better of targeting of anti-TNF therapy to patients early in their disease, when maximum effect can be achieved, and may result in greater access to these drugs as they are used in a more cost-efficient manner.

Some of the biomarkers described herein have previously been identified as associated with favourable response to anti-TNF therapy. The present invention is advantageous in enabling non-responders to be identified, so that such non-responders may be provided alternative treatment, and those who are not non-responders (and therefore may be a moderate or good responder) may be provided anti-TNF therapy. By identifying non-responders, both moderate and good responders are identified by subtraction as suitable for anti-TNF therapy compared to previous methods of prediction where only “good” responders may be identified thus failing to identify moderate responders who may also benefit. As a result of the present invention, anti-TNF therapies may therefore be used in a more targeted and cost-efficient manner.

The present invention provides an improved method for prediction of response to anti-TNF therapy, using biomarkers which could not have been predicted from the prior art as being indicative of non-favourable response and further biomarkers indicative of a favourable response.

The terms patient and subject are used interchangeably herein to refer to an individual for whom it is desirable to determine likely response to anti-TNF therapy.

Such an individual may have, or be predisposed to having, or expected to develop, an autoimmune or immune-mediated disorder.

A biomarker as used herein is a biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. A biomarker may be a gene, exhibiting differential expression between responders and non-responders to anti-TNF therapy. Expression of a biomarker gene (transcription and optionally translation) may be determined by measuring an expression product of the gene, referred to herein as a target molecule. A combination of two or more biomarkers may be referred to herein as a panel or a genetic signature which correlates with likely response to anti-TNF therapy.

An autoimmune or immune-mediated disorder as defined herein may include without limitation, Rheumatoid Arthritis, Ankylosing spondylitis, psoriatic arthritis, Behget's syndrome, inflammatory bowel disease, vasculitis, juvenile dermatomyositis, scleroderma, juvenile idiopathic arthritis, Crohn's disease, ulcerative colitis, psoriasis and systemic lupus erythematous.

Anti-TNF therapy is treatment which inhibits TNF activity, preferably directly, for example by inhibiting interaction of TNF with a cell surface receptor for TNF, inhibiting TNF protein production, inhibiting TNF gene expression, inhibiting TNF secretion from cells, inhibiting TNF receptor signalling or any other means resulting in decreased TNF activity in a subject. Anti-TNF therapy may also be referred to as TNF-inhibitory (TNFi) therapy. Anti-TNF therapeutics may be referred to as TNF inhibitors or antagonists and may encompass proteins, antibodies, antibody fragments, fusion proteins (e.g., Ig fusion proteins or Fc fusion proteins), multivalent binding proteins (e.g., DVD Ig), small molecule TNF antagonists and similar naturally- or non-naturally-occurring molecules, and/or recombinant and/or engineered forms thereof which inhibit TNF as described above, and in particular eliminate abnormal B cell activity. Anti-TNF therapy may include monoclonal antibodies such as infliximab (Remicade), adalimumab (Humira), certolizumab pegol (Cimzia), and golimumab (Simponi); circulating receptor fusion protein such as etanercept (Enbrel), together with functional equivalents, biosimilars or intended copies of these drugs and simple molecules such as xanthine derivatives (e.g. pentoxifylline and Bupropion.

Predicting response means making a determination of the likely effect of treatment in a subject. Prediction typically means an assessment made prior to commencing the relevant treatment, although it is understood that a prediction of the likely response to a particular treatment may be made whilst a subject is receiving an alternative treatment. Predicting response to therapy, within the scope of the present invention may also include making an assessment of likely continued response to anti-TNF therapy. Therefore, prediction of response may include a determination of likely response during a course of anti-TNF therapy.

A sample may be selected from the group comprising tissue sample, such as a biopsy sample; and a body fluid sample. A body fluid sample may be a blood sample. A blood sample may be a peripheral blood sample. It may be a whole blood sample, or cellular extract thereof. It may be a white blood cell fraction or neutrophil fraction of a blood sample. In a further embodiment, the sample is a purified neutrophil fraction.

The level of a target molecule herein refers to a measure of the amount of a target molecule in a sample. The level may be based upon a measure of one type of target molecule indicative of expression specific for a particular biomarker (i.e. DNA, RNA or protein). The level may alternatively be based upon a measure of a combination of two or more types of target molecule indicative of expression specific for a particular biomarker (i.e. two or more of DNA, RNA and protein). The level of a target molecule may be expressed as a direct measure of the amount of target molecule (for example concentration (mg/vol sample) or RPKM).

Elevated level means an increase in level (i.e. amount) of a target molecule compared to the level of the same target molecule in a subject who does not have an autoimmune or immune-mediated disorder (a control sample). An elevated level includes any statistically significant increase compared to the control. The level of a target molecule indicative of expression of a biomarker in a subject which does not have an auto-immune or immune mediated disorder may be referred to as a reference value or baseline value.

The elevated level of the target molecule representative of gene expression may be assessed by comparing the amount of the target molecule present in the patient sample under investigation with a reference value indicative of the amount of the target molecule in a control sample.

References herein to the “same” level of target molecule or biomarker expression indicate that the biomarker expression of the sample is identical to the reference or baseline value. References herein to a “similar” level of target molecule or biomarker expression indicate that the biomarker expression of the sample is not identical to the reference or baseline value but the difference between them is not statistically significant i.e. the levels have comparable quantities.

Suitable control samples for determination of a reference value or baseline value may be derived from individuals without an autoimmune or immune mediated disorder. Such an individual may be without the specific autoimmune or immune mediated disorder of the subject being tested, or more preferably may be without any autoimmune or immune mediated disorder. A control sample may be may be age matched with the patient undergoing investigation. Reference values or baseline value may be obtained from suitable individuals and used as a general reference value for multiple analysis.

Favourable response to anti-TNF therapy may include, without limitation, a reduction in pain, inflammation, swelling, stiffness, an increase in mobility, decreased time to disease progression, increased time of remission, improvement in function, improvement in quality of life. In rheumatoid arthritis, a favourable response may also include decreased progression of bone damage. In rheumatoid arthritis, a favourable response may be defined as a subject having a change in DAS28 of greater than or equal to 0.8, preferably greater than or equal to 1, and more preferably greater than or equal to 1.2 at week 12 after commencing anti-TNF therapy. A favourable response may further be defined as having a DAS28 of less than or equal to 3.2 at week 12 after commencing anti-TNF therapy.

A non-favourable response to anti-TNF therapy can include, without limitation, an increase or no improvement in pain, inflammation, swelling, stiffness, a decrease or no change in mobility, increased or no change in time to progression, increased or no change in time of remission, no increase in function or no improvement in quality of life. In rheumatoid arthritis, a non-favourable response may also include increased or no change in bone damage. In rheumatoid arthritis, a non-favourable response may be defined as a subject having a change in DAS28 of less than or equal to 1, less than or equal to 1.2 or less than or equal to 1.5 at week 12 after commencing anti-TNF therapy.

Activity of disease may include remission, progression or severity of disease, for example. Methods for determining disease activity will be available in the art and may be used in an embodiment. For Rheumatoid Arthritis, for example, many tools are available including Disease Activity Score of 28 joints (DAS28). From this, the disease activity of the affected person can be classified as follows:

| | | DAS28 decrease from initial value | | |
|---|---|---|---|---|
| Current DAS28 | | ≥1.2 | >0.6 but ≤1.2 | ≤0.6 |
| ≤3.2 | Inactive | Good | Moderate | No |
| | | improvement | improvement | improvement |
| ≥3.2 but | Moderate | Moderate | Moderate | No |
| ≤5.1 | | improvement | improvement | improvement |
| >5.1 | Very active | Moderate | No | No |
| | | improvement | improvement | improvement |

Other tools to monitor remission in rheumatoid arthritis include ACR-EULAR Provisional Definition of Remission of Rheumatoid arthritis, Simplified Disease Activity Index (SDAI) and Clinical Disease Activity Index (CDAI). For other conditions, tools include PsARC (psoriatic arthritis), PASI (Psoriasis), BASDAI (ankylosing spondylitis).

Target molecules as used herein may be selected from the group consisting of: a biomarker protein; and nucleic acid encoding the biomarker protein. The nucleic acid may be DNA or RNA. In an embodiment the nucleic acid is mRNA. Reference herein to a target molecule may include one type of biological molecule (i.e. DNA or RNA or protein) or a combination of two or more types of such biological molecules, all indicative of the expression of the same biomarker.

A binding partner may be selected from the group comprising: complementary nucleic acids: aptamers; receptors, antibodies or antibody fragments. By a specific binding partner is meant a binding partner capable of binding to at least one such target molecule in a manner that can be distinguished from non-specific binding to molecules that are not target molecules. A suitable distinction may, for example, be based on distinguishable differences in the magnitude of such binding.

The present invention provides for analysing an elevation occurring across a sum of biomarkers investigated. Analysis may be performed through relatively simple means, or may be undertaken using more complex algorithms. Examples of well-known and freely available software that can be used for the analysis of results relating to expression of target molecules in the methods of the invention are described in the paragraphs below. Preferred methods by which analysis of the results achieved may be undertaken may give rise to further useful aspects and embodiments of the invention.

By an LDG gene is meant a gene specifically expressed by a LDG cell. An LDG gene may be selected from the group consisting of AZU1, BPI, CEACAM8, CRISP3, CTSG, DEFA4, ELANE, LCN2, LTF, MMP8, MPO, RNASE2, RNASE3.

By an interferon related gene is meant a gene which encodes an expression product involved in the interferon signaling pathway. Herein, an interferon related gene may be selected from the group consisting of CMPK2, IFFI44L, IFI6, IFIT1B, LY6E, OAS1, OAS2, OAS3, RSAD2, and USP18.

For the purposes of the present disclosure the following protein nomenclature has been used:

AZU1 is a gene encoding azurocidin, which is an azurophil granulocyte antibiotic protein, also known as cationic antimicrobial protein or heparin binding protein;

BPI is a gene encoding the transcription factor Bactericidal/Permeability Increasing Protein.

CEACAM8 is a gene encoding Carcinoembryonic antigen-related cell adhesion molecule 8 (CEACAM8) also known as CD66b (Cluster of Differentiation 66b).

CRISP3 is a gene encoding Cysteine-rich secretory protein 3.

CTSG is a gene which encodes cathepsin G, also known as CG and CATG.

DEFA4 a gene which encodes Defensin, alpha 4 (DEFA4), also known as neutrophil defensin 4 or HNP4.

ELANE is a gene encoding an elastase, also known as neutrophil elastase; GE; NE; HLE; HNE: ELA2; SCN1; PMN-E.

LCN2 is a gene encoding Lipocalin-2 (LCN2), also known as oncogene 24p3 or neutrophil gelatinase-associated lipocalin (NGAL).

LTF is a gene encoding lactotransferrin, also referred to as HLF2; GIG12; and HEL110

MMP8 is a gene encoding matrix metalloproteinase-8, also known as neutrophil collagenase, PMNL collagenase (MNL-CL).

MPO is a gene encoding Myeloperoxidase.

RNASE2 is a gene encoding a RNase A Family, 2 (Liver, Eosinophil-Derived Neurotoxin). It may also be known as RNS2, EDN, Eosinophil-Derived Neurotoxin, Ribonuclease US, Ribonuclease 2, RNase Upl-2, EC 3.1.27.5, Non-Secretory Ribonuclease, Ribonuclease A F3 and RAF3.

RNASE3 is a gene encoding Ribonuclease, RNase A Family, 3, also referred to as RNS3, ECP, Eosinophil Cationic Protein, Ribonuclease 3, RNase 3, Cytotoxic Ribonuclease, EC 3.1.27.5, EC 3.1.27, EC 3.1.27

CMPK2 is a gene encoding Cytidine Monophosphate (UMP-CMP) Kinase 2, also referred to as Nucleoside-Diphosphate Kinase, Cytidylate Kinase 2, Thymidylate Kinase Family LPS-Inducible Member, Thymidine Monophosphate Kinase 2; UMP-CMP Kinase 2, Mitochondrial UMP-CMP Kinase, EC 2.7.4.14, EC 2.7.4.6, UMP-CMPK2, TMPK2, and TYKi.

IFFI44L is a gene encoding Interferon-Induced Protein 44-Like; also referred to as C1orf29, Chromosome 1 Open Reading Frame 29, and GS3686.

IF16 is a gene encoding Interferon, Alpha-Inducible Protein 6 also referred to as G1P3, Interferon-Induced Protein 6-16, IFI-6-16, IF1616, FAM14C and 6-16.

IFIT1 B is a gene encoding Interferon-Induced Protein With Tetratricopeptide Repeats 1B, also referred to as Interferon-Induced Protein With Tetratricopeptide Repeats 1-Like Protein, IFIT1L and BA149123.6.

LY6E is a gene encoding Lymphocyte Antigen 6 Complex, also referred to as Locus E, RIGE, SCA2, Retinoic Acid-Induced Gene E Protein, Retinoic Acid Induced Gene E, Thymic Shared Antigen 1, Stem Cell Antigen 2, Ly-6E, RIG-E, SCA-2, TSA-1, Lymphocyte Antigen 6E, 9804 and TSA1

OAS1 a gene encoding 2′-5′-Oligoadenylate Synthetase 1 also referred to as OIAS, 2-5-Oligoadenylate Synthetase 1, (2-5)Oligo(A) Synthase 1, 2-5A Synthase 1, P46/P42 OAS, E18/E16, 2-5 Oligoadenylate Synthetase 1 P48 Isoform, 2-5 Oligoadenylate Synthetase 1 P52 Isoform, 2,5-Oligoadenylate Synthetase 1 (40-46 KD), 2,5-Oligoadenylate Synthetase 1, 40/46 kDa, 2-5-Oligoisoadenylate Synthetase 1, 2-5-Oligoadenylate Synthase 1, (2-5)Oligo(A) Synthetase 1, 2,5-Oligo A Synthetase 1, 2-5A Synthetase 1, EC 2.7.7.84, EC 2.7.7, IFI-4, and OIASI

OAS2 is a gene encoding 2′-5′-Oligoadenylate Synthetase 2 also referred to as 2-5-Oligoadenylate Synthetase 2, 2-5-Oligoadenylate Synthetase 2 (69-71 KD), (2-5)Oligo(A) Synthase 2, P69 OAS/P71 OAS, P690AS/P71OAS, 2-5A Synthase 2, EC 2.7.7.84, and EC 2.7.7.

OAS3 is a gene encoding 2′-5′-Oligoadenylate Synthetase 3, also referred to as (2-5)Oligo(A) Synthase 3, 2-5A Synthase 3, P100 OAS, P100 OAS, 2-5-Oligoadenylate Synthetase 3 (100 KD), and 2-5 Oligoadenylate Synthetase P100, 2-5-Oligoadenylate Synthase 3, (2-5)Oligo(A) Synthetase 3, 2-5A Synthetase 3, EC 2.7.7.84, EC 2.7.7 and P100.

RSAD2 is a gene encoding Radical S-Adenosyl Methionine Domain Containing 2 also referred to as Viperin, Virus Inhibitory Protein, Endoplasmic Reticulum-Associated, Interferon-Inducible, Cytomegalovirus-Induced Gene 5 Protein, Cig5, Radical S-Adenosyl Methionine Domain-Containing Protein 2, 2510004L01Rik, Cig33, and Vig1.

USP18 is a gene encoding Ubiquitin Specific Peptidase 18, also referred to as ISG43, ISG15-Specific-Processing Protease, Ubiquitin Specific Protease 18, 43 KDa ISG15-Specific Protease, Ubl Thioesterase 18. HUBP43, UBP43, Ubl Carboxyl-Terminal Hydrolase 18, Ubl Thiolesterase 18, EC 3.1.2.15 and EC 3.4.19.

The first aspect of the present invention may make use of one or more target molecules, each target molecule being indicative of the expression of a different biomarker selected from the group consisting of: AZU1, BPI, CEACAM8, CRISP3. CTSG, DEFA4, ELANE, LCN2, LTF, MMP8, MPO, RNASE2, RNASE3. The first aspect of the invention may make use of two or more, three or more, four or more, five or more, six or more, seven or more, eight or more, nine or more, ten or more, eleven or more, twelve or more, or thirteen target molecules, each being indicative of the expression of a different biomarker selected from the group consisting of: AZU1, BPI, CEACAM8, CRISP3, CTSG, DEFA4, ELANE, LCN2, LTF, MMP8, MPO, RNASE2, and RNASE3.

In an embodiment, the first aspect of the present invention may make use of a target molecule indicative of the expression of RNASE3.

In an embodiment, the first aspect of the present invention may make use of a target molecule indicative of the expression of RNASE2.

In an embodiment, the first aspect of the present invention may make use of two or more target molecules, each being indicative of the expression of a different biomarker, wherein the biomarkers are RNASE3 and RNASE2.

Therefore, the present invention identifies a gene expression signature based which identifies subjects who are unlikely to respond or are likely to respond to anti-TNF therapy. In an embodiment, the signature is characterized by up-regulation of at least two genes, specifically RNASE3 and RNASE2.

The second aspect of the present invention may make use of one or more target molecules, each target molecule being indicative of the expression of a different biomarker selected from the group consisting of: CMPK2, IF16, RSAD2, and USP18. The second aspect of the invention may make use of two or more, three or more, or four target molecules, each being indicative of the expression of a different biomarker selected from the group consisting of: CMPK2, IFI6, RSAD2, and USP18.

In an embodiment, the second aspect of the present invention may make use of a target molecule indicative of the expression of CMPK2.

In an embodiment, the second aspect of the present invention may make use of a target molecule indicative of the expression of IFI6.

In an embodiment, the second aspect of the present invention may make use of a target molecule indicative of the expression of RSAD2.

In an embodiment, the second aspect of the present invention may make use of a target molecule indicative of the expression of USP18.

In an embodiment, the second aspect of the present invention may make use of four or more target molecules, each being indicative of the expression of a different biomarker, wherein the biomarkers are CMPK2, IFI6, RSAD2, and USP18.

The second aspect may further comprise determining the level of one or more, two or more, three or more, four or more, five or more or six target molecules, each being indicative of the expression of a different biomarker selected from the group consisting of IFFI44L, LY6E, OAS1, OAS2. OAS3 and IFIT1B.

The third aspect of the invention provide a combined approach, comprising determining the level of a target molecule indicative of the expression of a biomarker associated with favourable response and a biomarker associated with a non-favourable response. The third aspect may comprise any embodiment of the first and second aspects of the invention as defined herein, or any combination of embodiments of the first and second aspects of the invention. In an embodiment, the biomarkers of the third aspect include CMPK2, IFI44L, IFIT1B and RNASE3. Therefore, in an embodiment, the present invention identifies a gene expression signature based which identifies subjects who are unlikely to respond or are likely to respond to anti-TNF therapy. In an embodiment, the signature is characterized by up-regulation of at least four genes, specifically CMPK2, IFI44L, IFIT1B and RNASE3.

In an embodiment of the third aspect, the present invention provides determining the level of i) a target molecule indicative of the expression of each of AZU1, BPI, CEACAM8, CRISP3, CTSG, DEFA4, ELANE, LCN2, LTF, MMP8, MPO, RNASE2, RNASE3 to provide a genetic signature predictive of non-response to anti-TNF therapy and ii) a target molecule indicative of the expression of each of CMPK2, IFI6, RSAD2, USP18, IFFI44L LY6E, OAS1, OAS2, OAS3 and IFIT1B to provide a genetic signature predictive of response to anti-TNF therapy.

In an embodiment, the present invention provides methods for predicting the response of a subject to an anti-TNF therapy, wherein the therapy is selected from the group consisting of a protein, antibody, antibody fragment, fusion proteins (e.g., Ig fusion proteins or Fc fusion proteins), multivalent binding protein (e.g., DVD Ig), small molecule TNF antagonist, naturally- or non-naturally-occurring TNF antagonist, and/or recombinant and/or engineered forms thereof which inhibit TNF. In an embodiment, the anti-TNF therapy may be selected from the group consisting of a monoclonal antibody such as infliximab (Remicade), adalimumab (Humira), certolizumab pegol (Cimzia), and golimumab (Simponi); a circulating receptor fusion protein such as etanercept (Enbrel); together with any functional equivalents, biosimilars or intended copies of these drugs; and a simple molecule such as a xanthine derivative (e.g. pentoxifylline and Bupropion). In a preferred embodiment, the anti-TNF therapy is a monoclonal antibody, preferably adalimumab or etanercept, or biosimilar versions thereof.

The methods of the invention may make use of a range of patient samples, as defined herein. In an embodiment, the present invention may make use of a peripheral blood sample. In an embodiment, the present invention may make use of a white blood cell fraction, preferably a neutrophil fraction. Such a cellular fraction of blood may be prepared using methods known and available in the art, for example centrifugation followed by resuspension in suitable media (e.g. RPMI). A suitable method for extraction of a neutrophil fraction from a whole blood sample may be Polymorphprep (Axis Shield), Flcoll-Paque (GE Healthcare) or EasySep Human Neutrophil enrichment kit (StemCell). In an embodiment, a method of the invention may comprise extracting a white blood cell fraction from a blood sample of a subject. In an embodiment, a method of the invention may comprise extracting a neutrophil fraction from a blood sample of a subject. The present inventors have found that performing biomarker expression analysis on a white blood cell sample from a subject enables improved categorisation of the subject as a good or non-responder to anti-TNF therapy. Therefore, a method of the present invention comprising the step of extracting a cellular fraction (e.g. white blood cells or neutrophils) from the sample may represent a preferred embodiment. The method of the invention may also include the step of obtaining a sample from a subject.

A method of the invention will preferably be carried out in vitro, but it will be appreciated that a method of the invention may also be carried out in vivo.

A level of a target molecule may be investigated using a binding partner for the target molecule. A binding partner may be specific for a target molecule. In the context of the present invention, a binding partner specific to a target molecule will be capable of binding to at least one such target molecule in a manner that can be distinguished from non-specific binding to molecules that are not target molecules. A suitable distinction may, for example, be based on distinguishable differences in the magnitude of such binding.

Reference to a protein target may include precursors or variants produced on translation of the transcripts produced when the gene is expressed. Therefore, where a protein undergoes modification between first translation and its mature form, the precursor and/or the mature protein may be used as suitable target molecules. As above, techniques by which protein target molecules may be preserved within a patient sample, thus facilitating its detection, will be well known to those skilled in the art. A protein target may be found with a cell of a patient sample, or may be secreted or released from the cell.

In embodiments of the present invention where the target molecule is a protein, a binding partner may be used to determine the level of the protein in a sample obtained from the subject. A suitable binding partner may be is selected from the group consisting of aptamers; receptors, and antibodies or antibody fragments. Suitable methods for determining the level of a protein in a sample are available in the art. For example, in certain embodiments of the methods or devices of the invention the binding partner is an antibody, or antibody fragment, and the detection of the target molecules utilises an immunological method. In certain embodiments of the methods or devices, the immunological method may be an enzyme-linked immunosorbent assay (ELISA) including variants such as sandwich ELISAs; radioimmuno assays (RIA); In other embodiments an immunological method may utilise a lateral flow device. Other suitable techniques may include multiplex assays such as Luminex or proteomic MRM or fluorescence activated cell sorting (FACS); chemiluminescence.

In certain embodiments, a binding partner may be labelled, for example using a reporter moiety such as a fluorophore, chromogenic substrate or chromogenic enzyme. Where it is desired that the invention will make use of reporter moieties, the reporter moieties may be directly attached to the binding partners. Examples of such embodiments include those utilising labelled antibodies. Alternatively, the reporter moieties may be attached to reporter molecules that interact with the binding partners. Examples of such embodiments include those utilising antibodies indirectly attached to a reporter moiety by means of biotin/avidin complex.

In embodiments where the target molecule is a nucleic acid, binding partners may be complementary nucleic acids and aptamers, for example provided in a microarray or chip. Methods for determining the level of a nucleic acid target molecule in a sample are available in the art. In an embodiment, a suitable target molecule representative of gene expression may comprise an RNA transcript translatable to yield a protein. mRNA of this sort will typically be found within a patient sample. In particular, the transcriptome of white blood cells, for example neutrophils, of a patient sample have been found to provide a biomarker signature with improved sensitivity and specificity for determining non-responders and/or good responders to anti-TNF therapy, and the use of mRNA and in particular the transcriptome may represent a preferred embodiment. Use of mRNA as the target molecule has advantages in that the assays for detecting mRNA (such as quantitative rtPCR or the like) tend to be cheaper than methods for detecting protein(such as ELISAs). mRNA assays can be more readily multiplexed, allowing for high throughput analysis; nucleic acids generally show greater stability than their protein counterparts; and processing of the sample to obtain and amplify nucleic acid is generally simpler than for protein.

Techniques by which mRNA may be collected, purified and amplified as necessary, are well known to those skilled in the art. In an embodiment, the present invention may make use of transcriptome analysis for determining biomarker expression. Suitable techniques for determining the level of RNA in a sample, for example by transcriptome analysis, may include hybridization techniques, for example by detecting binding to a nucleic acid library, quantitative PCR, and high throughput sequencing including tag based sequencing such as SAGE (serial analysis of gene expression) and RNA-seq.

The above examples are non-limiting, and the methods of the invention may make use of any appropriate assay by which the presence or elevated levels of a requisite target molecule may be detected. It will be appreciated that suitable assays may be determined with reference to the nature of the target molecule to be detected and/or the nature of the patient sample to be used.

Multiple samples may be processed simultaneously, sequentially or separately. Multiple samples may processed simultaneously, for example in a high throughput method.

A method which may represent a preferred embodiment of the present invention may comprise the steps of isolating the mRNA from the sample; performing reverse transcriptase to obtain cDNA; amplifying the cDNA population; sequencing the cDNA population. Such a method may further comprise fragmenting the mRNA population; ligating adaptors to the mRNA and attaching barcodes to the cDNA population.

Known methods for high throughput sequencing which may be useful in the present invention include Illumina HiSeq™, Ion Torrent™, and SOLiD™.

Nucleic acid target molecule expression levels are typically expressed as Reads per kilobase of exon model per million mapped reads, which is calculated as (number of mapped reads×1 kilobase×1 million mapped reads)/(length of transcript×number of total reads) (RPKM).

Where the present invention uses a quantitative PCR based method for determining the level of a nucleic acid target molecule, the present invention may provide a kit comprising one or more pairs of primers of Table 4. Optionally, the kit may further comprise one or more of a set of instructions for use, a chart providing reference or baseline values for at least the biomarker corresponding to the primer pairs of the kits; and reagents.

Once the amounts or concentrations of the target molecules in the patient sample have been determined, this information may be used as the basis of an assessment of the predicted response to anti-TNF therapy, which may, in turn, be used to suggest a suitable course of treatment for the patient. The assessment may be qualitative or quantitative.

An elevated level of a biomarker may include at least 10%, 15, 20, 30, 40 50, 60, 70, 80, 90 or 100% or more increase compared to the baseline or reference value level. In one embodiment, an elevated level may be 1 fold or more difference relative to the baseline or reference value, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15 or 20 or any ranges therebetween. In one embodiment, the higher level is between a 1 and 15 fold difference relative to the baseline level, such as between a 1.5 and 12 fold difference relative to the baseline level. In a further embodiment, the higher level is between a 1 and 7 fold difference relative to the baseline level. It is appreciated that elevation levels may differ from the same biomarker depending on the target molecule being used. Where nucleic acid and protein target molecules are used for any particular biomarker, an elevated level may be expressed individually for a target molecule, or may be expressed as a sum or average of the target molecules.

For example, the methods of the invention may determine whether a target molecule indicative of expression of RNASE3 is elevated by 0.75 fold, I fold, 1.2 fold or 1.5 fold or more; and/or whether RNASE2 is elevated by 0.75 fold, 1 fold or 1.2 fold or more. If one or more of these target molecules are determined to be elevated by the stated values, then the subject would be classified as a non-responder to anti-TNF therapy and should receive alternative treatment.

Additionally, or alternatively, the methods of the invention may determine whether a target molecule indicative of expression of CMPK2 is elevated by 1 fold, 1.5 fold, 1.75 fold or 2-fold or more; and/or whether IF16 is elevated by 1 fold, 1.5 fold, 1.75 fold or 2-fold fold or more; and/or whether RSAD2 is elevated by 1 fold, 1.5 fold, 1.75 fold or 2-fold or more; and/or whether USP18 is elevated by 1 fold, 1.5 fold, 1.75 fold or 2-fold or more. If one or more of these target molecules are determined to be elevated by the stated values, then the subject would be classified as a responder to anti-TNF therapy and should receive anti-TNF therapy treatment.

The invention may produce a quantitative output, based upon elevation values for a biomarker or a sum or biomarkers. Alternatively, the invention may provide a qualitative output, based on likely response, for example yes/no; elevated; non-elevated; responder/non-responder; good, moderate or low based on EULAR criteria, etc. Where the levels of two or more target molecules are determined, a composite score may be determined, which may be compared to a composite score of reference values for the same target molecules.

In certain embodiments the methods or devices of the invention may further involve investigating physiological measurements of the patient.

In a further embodiment, there is provided a method for treating a subject having an autoimmune or immune-mediated disorder, wherein it was previously determined (or previously estimated) that a target molecule indicative of the expression of a Low Density Granulocyte (LDG) gene was increased in a sample from the subject compared to a reference value, the method comprising administering an alternative to anti-TNF therapy to the subject.

In a further embodiment, there is provided a method for treating a subject having an autoimmune or immune-mediated disorder, wherein it was previously determined (or previously estimated) that a target molecule indicative of the expression of a biomarker selected from the group consisting of: AZU1, BPI, CEACAM8, CRISP3, CTSG, DEFA4, ELANE. LCN2, LTF, MMP8, MPO, RNASE2, RNASE3 was increased in a sample from the subject compared to a reference value, the method comprising administering an alternative to anti-TNF therapy to the subject.

In a further embodiment, there is provided a method for treating a subject having an autoimmune or immune-mediated disorder, wherein it was previously determined (or previously estimated) that a target molecule indicative of the expression one or more interferon regulated biomarkers selected from the group consisting of: CMPK2, IFI6, RSAD2, and USP18 was increased in a sample from the subject compared to the level of the target molecule in a sample from a subject without an autoimmune or immune-mediated disorder, the method comprising administering an anti-TNF therapy to the subject.

In a further embodiment, there is provided a method for treating a subject having an autoimmune or immune-mediated disorder, wherein it was previously determined (or previously estimated) that i) a target molecule indicative of the expression of a Low Density Granulocyte (LDG) gene were not increased in a sample from the subject compared to a reference value and ii) a target molecule indicative of the expression one or more interferon regulated biomarkers selected from the group consisting of: CMPK2, IFI6, RSAD2, USP18, IFFI44L LY6E, OAS1, OAS2, OAS3 and IFIT1B were increased in a sample from the subject compared to a reference value; the method comprising administering an anti-TNF therapy to the subject.

In a further embodiment, there is provided a method for treating a subject having an autoimmune or immune-mediated disorder, wherein it was previously determined (or previously estimated) that i) a target molecule indicative of the expression of each of AZU1, BPI, CEACAM8, CRISP3, CTSG, DEFA4, ELANE, LCN2, LTF, MMP8, MPO, RNASE2, RNASE3 were not increased in a sample from the subject compared to a reference value and ii) a target molecule indicative of the expression of each of CMPK2, IFI6, RSAD2, USP18, IFFI44L LY6E, OAS1, OAS2, OAS3 and IFIT1B were increased in a sample from the subject compared to a reference value; the method comprising administering an anti-TNF therapy to the subject.

In a further embodiment, there is provided a method for treating a subject having an autoimmune or immune-mediated disorder, wherein it was previously determined (or previously estimated) that i) a target molecule indicative of the expression of a Low Density Granulocyte (LDG) gene were increased in a sample from the subject compared to a reference value and ii) a target molecule indicative of the expression one or more interferon regulated biomarkers selected from the group consisting of: CMPK2, IFI6, RSAD2, USP18, IFFI44L LY6E, OAS1, OAS2, OAS3 and IFIT1B were not increased in a sample from the subject compared to a reference value; the method comprising administering an alternative to anti-TNF therapy to the subject.

In a further embodiment, there is provided a method for treating a subject having an autoimmune or immune-mediated disorder, wherein it was previously determined (or previously estimated) that i) a target molecule indicative of the expression of each of AZU1, BPI, CEACAM8, CRISP3, CTSG, DEFA4, ELANE, LCN2, LTF, MMP8, MPO, RNASE2, RNASE3 were not increased in a sample from the subject compared to a reference value and ii) a target molecule indicative of the expression of each of CMPK2, IFI6, RSAD2, USP18, IFFI44L LY6E. OAS1, OAS2, OAS3 and IFIT1B were increased in a sample from the subject compared to a reference value; the method comprising administering an alternative to anti-TNF therapy to the subject.

It is envisaged that in the methods of treating a subject as defined herein, the previous determination of the level of a target molecule may be as defined in any one of the first, second or third aspects and embodiments thereof.

In an embodiment, there is provided a method for monitoring response to therapy, the method comprising determining activity of the autoimmune or immune-mediated disorder, wherein it was previously predicted that the subject would have a favourable response to anti-TNF therapy, and wherein the patient has been administered anti-TNF therapy. It is envisaged that in such a method, the prediction of response to anti-TNF therapy was carried out in previous determination of the level of a target molecule may be as defined in any one of the first, second or third aspects and embodiments thereof.

The present invention may further provide a method of selecting a treatment regimen for a subject, comprising assaying a sample obtained from the subject, wherein the method comprises predicting whether the subject will be a responder or non-responder to anti-TNF therapy according to any one of the first, second or third aspects of the present invention, wherein an elevated level of a target molecule according to the first aspect indicates that the subject will benefit from an alternative treatment to anti-TNF therapy; wherein an elevated level of a target molecule according to the second aspect indicates that the subject will benefit from anti-TNF therapy.

In a further aspect, the present invention provides kits for use in the methods described herein. Such kits may comprise binding partners capable of binding to a target molecule. In the case of a protein target molecule, such binding partners may comprise antibodies that bind specifically to the protein. In the case of a nucleic acid target molecule the binding partner may comprise a nucleic acid complementary to the target molecule. In the case of a protein target molecule the kit may comprise antibody or antibody fragments specific for the target molecule. The kit may also comprise a set of instruction for use of the kit, and reference values for a control sample, in order to determine any elevation in target molecule in the sample.

It is envisaged that the embodiments of the aspects of the present invention apply to the other aspects of the invention, mutatis mutandis.

The use of the word “a” or “an” when used in conjunction with the term “comprising” in the claims and/or the specification may mean “one,” but it is also consistent with the meaning of “one or more,” “at least one,” and “one or more than one.”

The invention will now be further described by way of example and without limitation, with reference to the Tables 1 to 5 and the Figures.

### Examples

### Patients and Methods

Ethics Statement.

This study was approved by the University of Liverpool CORE (Committee on Research Ethics for healthy controls), and North West 3 (Liverpool East) Research Ethics Committee for RA patients. All participants gave written, informed consent.

Patients.

For the original study, twenty patients with RA and six healthy controls were recruited to the study. All patients fulfilled ACR criteria for RA [1], were Biologic naïve and about to receive therapy with a TNFi. Inclusion criteria were: 18-80 years of age, a failure of at least two disease modifying anti-rheumatic drugs (DMARDs) including methotrexate (MTX), and active disease (DAS28>5.1) according to NICE guidelines for prescribing biologic therapy for RA in the UK. For the validation study, thirty-two patients with RA (sixteen DMARD-naïve, and sixteen pre-TNFi) fulfilling the same criteria were recruited to the study. Patient clinical characteristics in each cohort, pre- and post therapy (weeks 0 and 12), are shown in Tables 1-3. The response to therapy was measured in two ways at week 12: a decrease in DAS28>1.2 (BSR guidelines) to define “Responder” or “Non-Responder”; or according to EULAR guidelines [1], by which response is defined as “Good”, “Moderate” or “None” according to the following criteria:—

| DAS28 | Improvement in DAS28 | | |
|---|---|---|---|
| endpoint | >1.2 | >0.6 and ≤1.2 | ≤0.6 |
| ≤3.2 | Good | | |
| >3.2 and | | Moderate | |
| ≤5.1 | | | |
| >5.1 | | | None |

Isolation of Neutrophils.

In the original study, blood (20 mL) was collected into lithium-heparin vacutainers from RA patients prior to commencement of therapy (week 0), or healthy controls. Neutrophils were isolated using Polymorphprep (Axis Shield), and contaminating erythrocytes were removed by hypotonic lysis. Neutrophils were resuspended at 5×10⁶ cells/mL in RPMI 1640 media plus HEPES (Gibco). In the validation study, 20 mL whole blood was mixed with HetaSep solution (StemCell) at a ratio of 1:5 (HetaSep: whole blood) and incubated at 37° C. for 30 min until the plasma/erythrocyte interphase was at approximately 50% of the total volume. The leukocyte-rich plasma layer was carefully removed and washed in a 4-fold volume of recommended media (Mg²⁺ and Ca²⁺-free PBS, +2% FBS and 1 mM EDTA). Cells were centrifuged at 200 g for 10 min and resuspended in a 4-fold volume of recommended media. Washed leukocytes were layered onto Ficoll-Paque (GE Healthcare) 1:1 and centrifuged at 500 g for 30 min. The PBMC layer was discarded, and the granulocyte pellet was resuspended in recommended media, centrifuged for 3 min at 500 g and resuspended in recommended media at a concentration of 5×10⁷ cells/mL. Highly pure neutrophils were isolated from the granulocyte pellet using the EasySep Human Neutrophil enrichment kit (StemCell), following the manufacturer's instructions. Briefly, 50 μL of EasySep® neutrophil enrichment cocktail, containing a mix of tetrameric antibody complexes produced from monoclonal antibodies (also bispecific for dextran) directed against the cell surface antigens CD2, CD3, CD9, CD19, CD36, CD56 and glycophorin A was added per 1 mL of nucleated cells and incubated for 10 min on ice. 100 μL of EasySep dextran-coated nanoparticle beads were added per 1 mL of nucleated cells and incubated for a further 10 min on ice. The cell/antibody/bead solution was adjusted to a total volume of 2.5 mL with recommended media and placed into an EasySep magnet for 5 min at room temperature. Unbound neutrophils were decanted and placed into an EasySep magnet for a further 5 min. Highly-pure, unbound neutrophils were briefly centrifuged and resuspended in RPMI 1640 media plus 25 mM HEPES to a concentration of 5×10⁶/mL.

Isolation of RNA.

RNA was isolated from a minimum of 10⁷ cells using Trizol-chloroform (Invitrogen), precipitated in isopropanol and cleaned using the RNeasy kit (Qiagen) including a DNase digestion step. Total RNA concentration and integrity were assessed using the Agilent 2100 Bioanalyser RNA Nano chip. RNA integrity (RIN) was routinely found to be ≥7.0.

RNA-Seq Library Generation and Sequencing.

Total RNA was enriched for mRNA using poly-A selection. Standard Illumina protocols were used to generate 50 base pair single-end read libraries. Briefly, mRNA was fragmented, reverse transcribed, adapted with sequencing primers and sample barcodes, size selected and PCR-enriched. Libraries were sequenced on the Illumina HiSeq 2000 platform.

Read Mapping and Gene Annotation.

Reads were mapped to the human genome (hg19) using TopHat v2.0.4 [2] applying the—max-multihits 1 setting. Count data was generated using HTSeq v0.5 [3] and gene expression (RPKM) [4] values were calculated using Cufflinks v2.0.2 [2]. A minimum RPKM threshold of expression of a 0.3 was applied to the data in order to minimise the risk of including false positives against discarding true positives from the dataset [5-7].

Bioinformatics.

Bioinformatics analysis was carried out using IPA (Ingenuity® Systems, www.ingenuity.com), which identified the pathways from the IPA library of canonical pathways that were most significantly represented in the dataset.

qPCR Analysis.

cDNA was synthesised from total RNA using the Superscript III First Strand cDNA Synthesis kit (Invitrogen) using equal concentrations of RNA across samples, as per the manufacturer's instructions. Real-time PCR analysis was carried out using the QuantiTect SYBR Green PCR kit (Qiagen) as per the manufacturer's instructions. Analysis was carried out on a Roche 480 LightCycler in a 96-well plate using a 20 μL reaction volume. Target gene expression was quantified using Mean Normalised Expression against B2M as a housekeeping gene[8]. Primer sequences can be found in Table 4.

Statistical Analysis.

Statistical analysis of RNA-Seq count data was carried out using edgeR v3.0.8 [9] with a 5% false discovery rate (FDR). Binary logistic regression and Receiver Operating Characteristic (ROC) area under the curve (AUC) methodology was used on each of the individual 23 biomarker genes, and then collectively, to find optimum panels for prediction of response to TNFi.

### Results

Analysis of Genes with Significantly Different Expression Levels in TNFi Responders and Non-Responders

In order to identify genes with significantly different expression (DE) levels in TNFi responders and non-responders from the original cohort, we ran edgeR analysis on RNA-Seq count data, classifying patients as responders or non responders based on the change in DAS28 from week 0 to week 12. A decrease in DAS28<1.2 was classed as non-response to TNFi. Applying an FDR<0.05 we identified 47 genes with significantly DE levels prior to commencing TNFi in each patient group (Table 5). Of these genes, 11 were higher in responders and 36 were higher in non-responders.

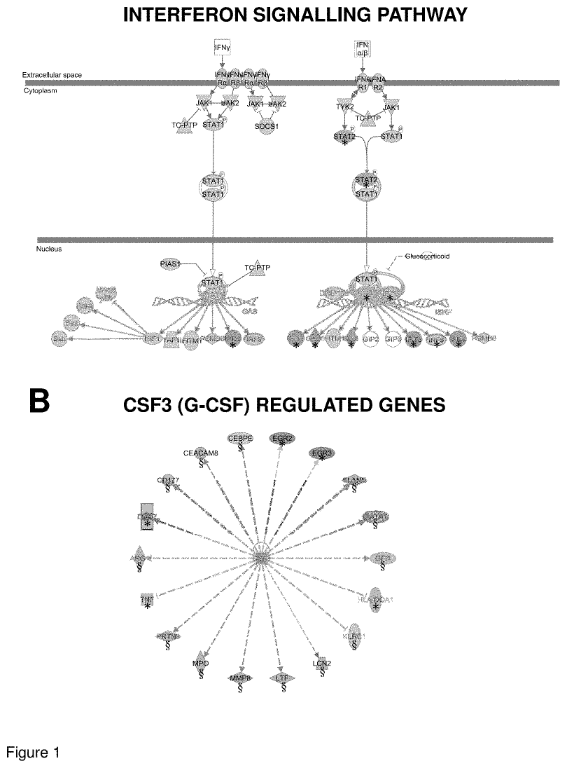

We used Ingenuity (IPA) analysis of the significantly DE genes to identify Interferon Signalling as the most enriched pathway in the TNFi responders (p=0.0001, FIG. 1A) [10]. Ingenuity also predicted that interferons were acting as upstream regulators in the TNFi responders (IFNA2 p=2.49×10⁻²⁹, z-score=6.594; IFNG p=6.22×10⁻²⁶, z-score 5.196). Of the 11 genes significantly DE in TNFi responders 10 were predicted to be regulated by IFNs: CMPK2, IFI44L, IFIT1B, IF16, LY6E, OAS1, OAS2, OAS3, RSAD2, USP18.

IPA also predicted that CSF3 (G-CSF) was negatively regulating the gene expression in the TNFi responders, and conversely this means that G-CSF was positively regulating the gene expression in the TNFi non-responders (p=1.4×10⁻⁶, z-score=−2.609, FIG. 1B). We observed that the genes with which IPA was able to make this prediction closely resembled the expression profile of Low Density Granulocytes (LDGs, immature neutrophils) previously identified in Systemic Lupus Erythematosus patients [11]. Of the 47 non-responder genes identified as significantly DE by the edgeR analysis, 13 directly related to LDG genes being expressed in RA neutrophils: AZU1, BPI, CEACAM8, CRISP3, CTSG, DEFA4, ELANE, LCN2, LTF, MMP8, MPO, RNASE2, RNASE3.

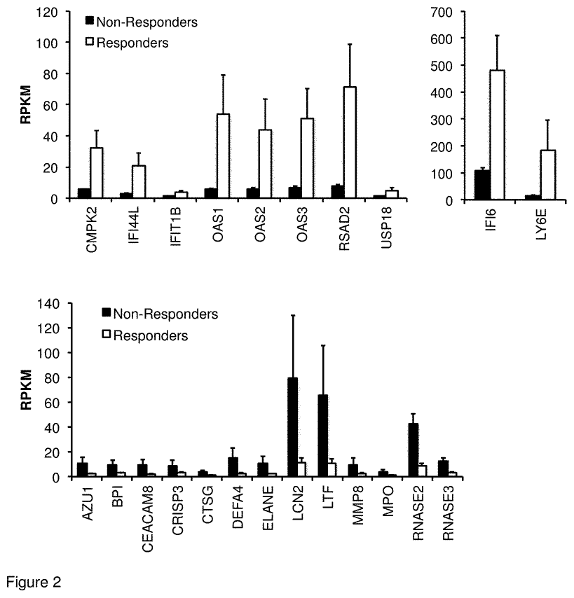

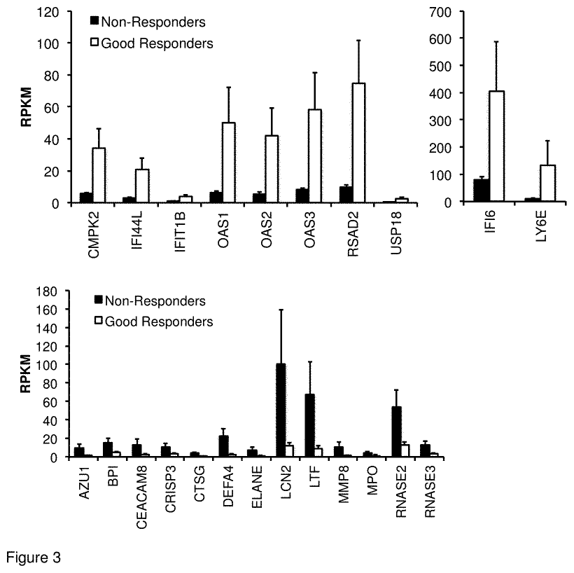

The level of expression of the 10 IFN-regulated genes and 13 LDG-genes significantly DE between TNFi responders and non-responders are shown in FIG. 2. Response is classified as a decrease in DAS28>1.2. In FIG. 3 the expression levels of the 10 IFN-regulated genes and 13 LDG-genes are shown only for those patients achieving a EULAR “Good” or “None” Response [1]. In this context, a EULAR “Good” response is classified as a decrease in DAS28>1.2 and a DAS28 endpoint≤3.2 as defined above.

### Validation of IFN and LDG Gene Expression Profiles in Separate Cohort of RA Patients

In order to validate the expression level of the IFN and LDG genes in TNFi responders and non-responders we recruited two validation cohorts of patients: 16 early arthritis (pre-DMARD patients) and 16 Biologic naïve (pre-TNFi) patients. RNA was extracted from highly-pure peripheral blood neutrophils, and the expression levels of 10 IFN-regulated and 13 LDG marker genes were measured using qPCR (normalised to B2M housekeeping gene using Mean Normal Expression, MNE).

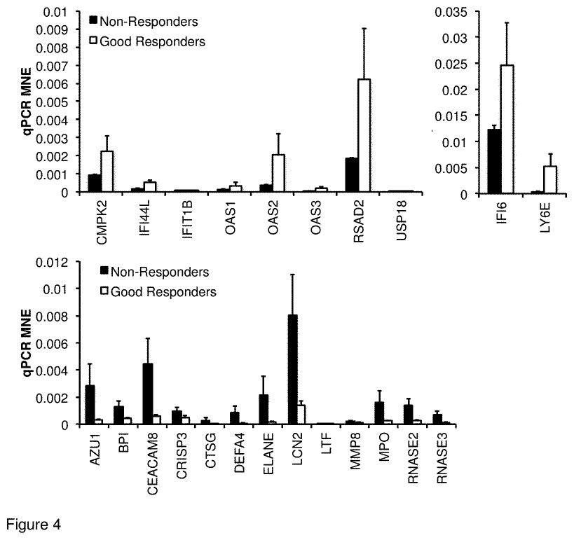

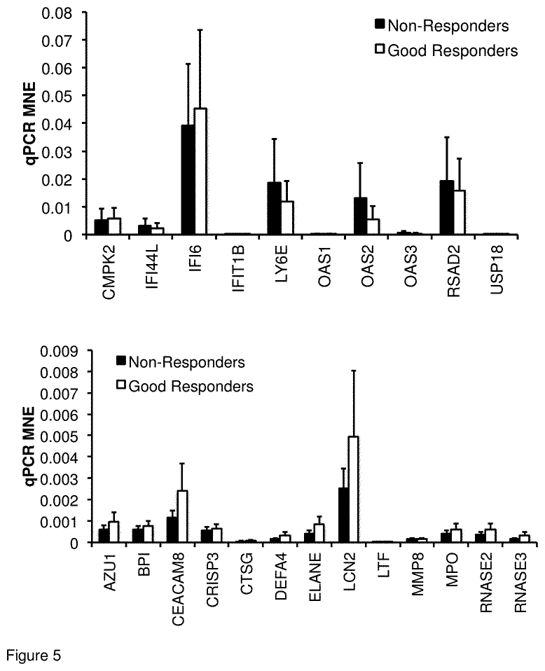

Expression levels of the IFN and LDG genes in the validation cohort followed the same expression profile in TNFi responders and non-responders as the initial cohort (FIG. 4). However there was no association of expression level with response to DMARDs (FIG. 5) confirming that these biomarker genes are specific for response to TNFi.

### Statistical Analysis of Biomarker Genes for Prediction of Response to TNFi

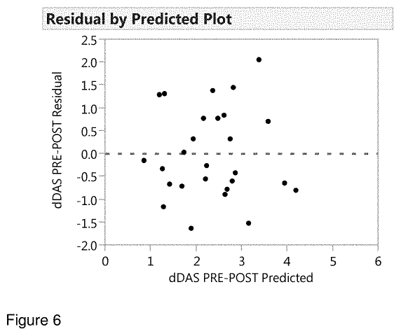

Binary logistic regression and ROC AUC methodology was used on each of the individual 23 biomarker genes, and then collectively to find optimum panels for prediction. The results of this analysis are shown in Table 6. Stepwise regression of the 23 genes to find a good subset of predictors identified CMPK2, IFI44L, IFIT1B and RNASE3 (FIG. 6) as the optimal combination of predictor genes.

Response dDAS PRE-POST

### Summary of Fit

| | |
|---|---|
| RSquare | 0.437291 |
| RSquare Adj | 0.33498 |
| Root Mean Square Error | 1.0661 |
| Mean of Response | 2.350741 |
| Observations (or Sum Wgts) | 27 |

### Analysis of Variance

| | | Sum of | Mean | |
|---|---|---|---|---|
| Source | DF | Squares | Square | F Ratio |
| Model | 4 | 19.431450 | 4.85786 | 4.2741 |
| Error | 22 | 25.004535 | 1.13657 | Prob > F |
| C. Total | 26 | 44.435985 | | 0.0104* |

### Parameter Estimates

| Term | Estimate | Std Error | t Ratio | Prob > \|t\| |
|---|---|---|---|---|
| Intercept | 5.7796476 | 1.356228 | 4.26 | 0.0003* |
| logCMPK2 | −2.315782 | 1.002302 | −2.31 | 0.0306* |
| logIFI44L | 1.0426791 | 0.7292.25 | 1.43 | 0.1668 |
| logIFIT1B | 1.7346525 | 0.84565 | 2.05 | 0.0523 |
| logRNASE3 | −0.499906 | 0.213394 | −2.34 | 0.0286* |

### Discussion

The study demonstrates that expression of 23 genes in peripheral blood neutrophils from rheumatoid arthritis patients can predict response to TNFi (as a first Biologic). Expression of an IFN-regulated gene expression signature comprising 10 genes (CMPK2, IF144L, IFIT1B, IFI6, LY6E, OAS1, OAS2, OAS3, RSAD2, USP18) predicts response to TNFi, and expression of LDG genes (AZU1, BPI, CEACAM8, CRISP3, CTSG. DEFA4, ELANE, LCN2, LTF, MMP8, MPO. RNASE2, RNASE3) by mature neutrophils predicts non-response to TNFi. These two gene expression profiles are mutually exclusive and therefore comprise a biomarker panel with a high degree of sensitivity and specificity for the prediction of response to TNFi in RA.

### Tables

TABLE 1
Clinical characteristics of 20 rheumatoid arthritis patients
in the original study prior to, and 12-weeks post, commencement
of TNFi therapy. Values given as mean (range).

| | Pre Therapy | | Post Therapy | |
|---|---|---|---|---|
| No. of patients | 20 | | | |
| Age (Y) | 53 | (36-76) | | |
| Disease duration (Y) | 10 | (0.5-25) | | |
| Sex: female, male | 17, 3 | | | |
| CRP (mg/L) | 32 | (6-212) | 22 | (0-191) |
| ESR (mm/h) | 39 | (5-73) | 30 | (2-94) |
| RF + ve | 17 | | | |
| DAS28 | 6.22 | (5.10-7.56) | 3.93 | (1.85-6.62) |

| | | |
|---|---|---|
| Therapy commenced | | |
| Adalimumab | 13 | |
| Etanercept | 5 | |
| Golimumab | 2 | |
| Change in DAS 28 | | |
| ≥1.2 | | 16 |
| <1.2 | | 4 |
| EULAR response | | |
| Good | | 5 |
| Moderate | | 13 |
| None | | 2 |

TABLE 2
Clinical characteristics of 16 rheumatoid arthritis patients
in the validation study prior to, and 12-weeks post, commencement
of TNFi therapy. Values given as mean (range).

| | Pre Therapy | | Post Therapy | |
|---|---|---|---|---|
| No. of patients | 16 | | | |
| Age (Y) | 60 | (38-73) | | |
| Disease duration (Y) | 12 | (1-30) | | |
| Sex: female, male | 12, 4 | | | |
| CRP (mg/L) | 16 | (<5-43) | 20 | (<5-62) |
| ESR (mm/h) | 36 | (8-83) | 37 | (2-85) |
| RF + ve | 12 | | | |
| DAS28 | 5.89 | (5.15-7.41) | 4.05 | (2.72-6.95) |

| | | |
|---|---|---|
| Therapy commenced | | |
| Adalimumab | 9 | |
| Etanercept | 4 | |
| Golimumab | 3 | |
| Change in DAS 28 | | |
| ≥1.2 | | 12 |
| <1.2 | | 4 |
| EULAR response | | |
| Good | | 5 |
| Moderate | | 8 |
| None | | 3 |

TABLE 3
Clinical characteristics of 16 early rheumatoid arthritis patients
in the validation study prior to, and 12-weeks post, commencement
of DMARD therapy. Values given as mean (range).

| | Pre Therapy | | Post Therapy | |
|---|---|---|---|---|
| No. of patients | 16 | | | |
| Age (Y) | 59 | (21-80) | | |
| Disease duration (Y) | 0 | (0) | | |
| Sex: female, male | 9, 7 | | | |
| CRP (mg/L) | 32 | (<5-149) | 27 | (<5-195) |
| ESR (mm/h) | 54 | (10-129) | 35 | (4-138) |
| RF + ve | 11 | | | |
| DAS28 | 5.89 | (4.06-7.92) | 3.79 | (1.54-7.09) |

| | | |
|---|---|---|
| Therapy commenced | | |
| MTX | 15 | |
| HCQ | 15 | |
| SSZ | 4 | |
| Change in DAS 28 | | |
| ≥1.2 | | 11 |
| <1.2 | | 5 |
| EULAR response | | |
| Good | | 5 |
| Moderate | | 7 |
| None | | 4 |

TABLE 4
List of primers used in qPCR
validation of biomarker gene expression

| gene | Sequence |
|---|---|
| AZU1-F | CAGCAGCATGAGCGAGAATG |
| AZU1-R | AGAGGCAGTGGCAGTATCGT |
| B2M-F | ACTGAATTCACCCCCACTGA |
| B2M-R | CCTCCATGATGCTGCTTACA |
| BPI-F | GGCATGCACACAACTGGTTC |
| BPI-R | CCAGCTTGAGCTCTCCAACA |
| CEACAM8-F | GCGGAACGTCACCAGAAATG |
| CEACAM8-R | GAGTCTCCGGATGTACGCTG |
| CMPK2-F | AGGCCAACAGTGTGTTTCGT |
| CMPK2-R | ACCGTCTGCAGGACCTTTTC |
| CRISP3-F | TCTGGAAACCACTGCAATGAC |
| CRISP3-R | AGCAGTAAAAGCGGGATCCTT |
| CTSG-F | TCCGCATCTTCGGTTCCTAC |
| CTSG-R | CGTGGGCCACATTGTTACAC |
| DEFA4-F | CTGCCTCATTGGTGGTGTGA |
| DEFA4-R | GGCGTTCCCAGCATGACATT |
| ELANE-F | CGTGGCGAATGTAAACGTCC |
| ELANE-R | TTTTCGAAGATGCGCTGCAC |
| IFI44L-F | CCTAGCCATGTGTCCTTCCA |
| IFI44L-R | GCTTTCACAGCTAGTAAGAGGACT |
| IFI6-F | CAAGGTCTAGTGACGGAGCC |
| IFI6-R | TTTCTTACCTGCCTCCACCC |
| IFIT1B-F | TACTGGGTACGCAATCACCG |
| IFIT1B-R | GCTCGTTTTAGGACGTGCAG |
| LCN2-F | CAGGACTCCACCTCAGACCT |
| LCN2-R | CTGCCAGGCCTACCACATAC |
| LTF-F | TTTTCGGAGCCTGGATCCTC |
| LTF-R | CGCCCCTTTATTCAGGGCTT |
| LY6E-F | AGGACAGGCTGCTTTGGTTT |
| LY6E-R | AGCAGCACTGGCAAGAAGAT |
| MMP8-F | CCTTGCTAAGGACTACTGGGC |
| MMP8-R | CTGGCCCATTTGGGTTTGGA |
| MPO-F | ATCGCCAACGTCTTCACCAA |
| MPO-R | CATGGGCTGGTACCGATTGT |
| OAS1-F | GATTCTGCTGGCTGAAAGCAA |
| OAS1-R | GAGTGTGCTGGGTCTATGAG |
| OAS2-F | CTCCTCCTTTTTCCTTCCAGTCT |
| OAS2-R | AAGCACCGAGAGCAAGATCA |
| OAS3-F | TGGACCATCAACTACAACGCC |
| OAS3-R | ATCCAGGATGATAGGCCTGAACC |
| RNASE2-F | GTGGAAGCCAGGTGCCTTTA |
| RNASE2-R | CATGTTTGCTGGTGTCTGCG |
| RNASE3-F | GCACGTATGCAGACAGACCA |
| RNASE3-R | GGTGAACTGGAACCACAGGA |
| RSAD2-F | TGCTGGGAAGCTCTTGAGTG |
| RSAD2-R | AGCTAGCAGCCAGAAGGTTG |
| USP18-F | AGTCCCCGGCAGATCTTGAA |
| USP18-R | AAACCAACCAGGCCATGAGG |

TABLE 5
Genes with significantly different expression levels between TNFi responders and non-responders in the original cohort,
identified by edgeR analysis of RNA-Seq count data (FDR <0.05).

| | | | | | | | Non- | |
|---|---|---|---|---|---|---|---|---|
| | | | | | | Responder | Responder | Validation |
| GeneID | Name | logFC | logCPM | PValue | FDR | Gene | Gene | Set |
| ABCA13 | ATP-binding cassette. sub-family A | −1.894 | 1.8067 | 0 | 0.002 | | X | |
| | (ABC1), member 1 | | | | | | | |
| ANLN | anillin, actin binding protein | −1.5874 | −1.1368 | 0.0001 | 0.0285 | | X | |
| ANXA1 | annexin A1 | −1.028 | 6.8764 | 0.0001 | 0.0443 | | X | |
| AZU1 | azurocidin 1 | −2.3117 | 1.3057 | 0 | 0.0005 | | X | X |
| BAIAP3 | BAI1-associated protein 3 | −1.117 | 5.3063 | 0.0001 | 0.0332 | | X | |
| BCAT1 | branched chain aminotransferase 1, | −1.9977 | 2.3029 | 0.0001 | 0.0443 | | X | |
| | cytosolic | | | | | | | |
| BPI | bactericidal/permeability-increasing | −1.6853 | 3.3115 | 0 | 0.0135 | | X | X |
| | protein | | | | | | | |
| C5orf30 | chromosome 5 open reading frame | −1.0607 | 1.7366 | 0.0001 | 0.0362 | | X | |
| CD24 | CD24 molecule; CD24 molecule-like 4 | −1.5264 | 4.186 | 0 | 0.0007 | | X | |
| CDHR2 | protocadherin 24 | −1.7411 | −0.0569 | 0 | 0.012 | | X | |
| CEACAM8 | carcinoembryonic antigen-related cell | −2.6204 | 3.3309 | 0 | 0.0002 | | X | X |
| | adhesion molecule 8 | | | | | | | |
| CH1T1 | chitinase 1 chitotriosidase | −2.0599 | 1.7354 | 0 | 0.0035 | | X | |
| CMPK2 | cytidine monophosphate (UMP-CMP) | 2.9451 | 6.1137 | 0.0001 | 0.0403 | X | | X |
| | kinase 2, mitochondrial | | | | | | | |
| CRISP3 | cysteine-rich secretory protein 3 | −1.6316 | 3.2048 | 0.0001 | 0.0443 | | X | X |
| CTSG | cathepsin G | −2.454 | −0.2512 | 0 | 0.0014 | | X | X |
| DEFA4 | defensin alpha 4, corticostatin | −2.8131 | 1.1418 | 0 | 0.0001 | | X | X |
| ECRP | Rnase A family 2 pseudogene | −2.0865 | 0.4211 | 0 | 0.0004 | | X | X |
| ELANE | elastase, neutrophil expressed | −2.4612 | 0.8798 | 0 | 0.0002 | | X | X |
| ERG | v-ets erythroblastosis virus E26 | −2.933 | −1.1458 | 0 | 0.0004 | | X | |
| | oncogene homolog (avian) | | | | | | | |
| HTRA3 | HtrA serine peptidase 3 | −2.8968 | −1.7752 | 0 | 0.0003 | | X | |
| IFI44L | interferon-induced protein 44-like | 3.4368 | 7.0578 | 0.0001 | 0.0403 | X | | X |
| IFI6 | interferon, alpha-inducible protein 6 | 2.3794 | 7.5956 | 0.0001 | 0.0255 | X | | X |
| IFIT1B | interferon-induced protein with | 2.555 | 2.6771 | 0.0002 | 0.0443 | X | | X |
| | tetratricopeptide repeats 1-like | | | | | | | |
| INHBA | inhibin, beta A | −1.9195 | −1.4092 | 0 | 0.0043 | | X | |
| LAMP3 | lysosomal-associated membrane | 2.0942 | 2.943 | 0.0002 | 0.0494 | X | | |
| | protein 3 | | | | | | | |
| LCN2 | lipocalin 2 | −2.7106 | 4.6133 | 0 | 0.0005 | | X | X |
| LTF | Lactotransferrin | −2.5121 | 5.9162 | 0 | 0.007 | | X | X |
| LY6E | lymphocyte antigen 6 complex, locus E | 3.8126 | 7.0927 | 0 | 0.0139 | X | | X |
| MAOA | monoamine oxidase A | −2.4712 | −2.048 | 0 | 0.0044 | | X | |
| MMP8 | matrix metallopeptidase 8 | −2.4325 | 3.569 | 0 | 0.0208 | | X | X |
| | (neutrophil collagenase) | | | | | | | |
| MPO | Myeloperoxidase | −2.6018 | 1.8822 | 0 | 0.0001 | | X | X |
| MS4A3 | membrane-spanning 4-domains, | −2.2529 | 1.9085 | 0 | 0.0005 | | X | |
| | subfamily A, member 3 | | | | | | | |
| | (hematopoietic cell-specific) | | | | | | | |
| MXRA7 | matrix-remodelling associated 7 | −1.636 | 3.6657 | 0.0002 | 0.046 | | X | |
| OAS1 | 2′,5′-oligoadenylate synthetase 1, | 3.6981 | 6.573 | 0.0001 | 0.0247 | X | | X |
| | 40/46 kDa | | | | | | | |
| OAS2 | 2′-5′-oligoadenylate synthetase 2, | 3.3233 | 7.3834 | 0.0001 | 0.0242 | X | | X |
| | 69/71 kDa | | | | | | | |
| OAS3 | 2′-5′-oligoadenylate synthetase 3, | 3.3085 | 8.5453 | 0 | 0.0155 | X | | X |
| | 100 kDa | | | | | | | |
| OLR1 | oxidized low density lipoprotein | −2.5139 | 0.5408 | 0 | 0.0041 | | X | |
| | (lectin-like) receptor 1 | | | | | | | |
| PDLIM1 | PDZ and LIM domain 1 | −1.269 | 2.6386 | 0.0002 | 0.046 | | X | |
| PRTN3 | proteinase 3 | −3.1239 | 0.3539 | 0 | 0.0001 | | | |
| RNASE2 | ribonuclease, RNase A family, 2 | −2.2537 | 4.0552 | 0 | 0 | | X | X |
| | (liver, eosinophil-derived neurotoxin) | | | | | | | |
| RNASE3 | ribonuclease, RNase A family, 3 | −2.1538 | 1.7063 | 0 | 0.0001 | | X | X |
| | (eosinophil cationic protein) | | | | | | | |
| RSAD2 | radical S-adenosyl methionine domain | 3.5236 | 7.8658 | 0.0001 | 0.0366 | X | | X |
| | containing 2 | | | | | | | |
| SAP30 | Sin3A-associated protein, 30 kDa | −1.782 | 1.7137 | 0 | 0.0005 | | X | |
| SERPINB10 | serpin peptidase inhibitor, Glade B | −2.3213 | −1.1613 | 0.0001 | 0.0272 | | X | |
| | (ovalbumin), member 10 | | | | | | | |
| SLC2A5 | solute carrier family 2 (facilitated | −2.4158 | −0.2374 | 0 | 0.0002 | | X | |
| | glucose/fructose transporter), member 5 | | | | | | | |
| USP18 | ubiquitin specific peptidase 18 | 3.512 | 2.5994 | 0.0002 | 0.046 | X | | X |
| ZNF608 | zinc finger protein 608 | −1.1443 | 3.6281 | 0.0001 | 0.0443 | | X | |

TABLE 6
Receiver Operator Characteristic (ROC) analysis for 10 IFN-
regulated genes and 13 LDG genes from the original cohort,
showing area under the curve (AUG), P-value, specificity and
sensitivity of each gene to predict “Good” or “No”
response to TNFi based on decrease in DAS28 from week 0 to week 12.

| Gene | ROC AUC | P-value | Specificity | Sensitivity |
|---|---|---|---|---|
| CMPK2 | 0.75 | 0.0499 | 100% | 75% |
| IFI44L | 0.72917 | 0.0304 | 100% | 62.5% |
| IFI6 | 0.70833 | 0.0832 | 100% | 62.5% |
| IFIT1B | 0.79167 | 0.0198 | 100% | 75% |
| LY6E | 0.68750 | 0.0581 | 100% | 50% |
| OAS1 | 0.7500 | 0.0251 | 100% | 62.5% |
| OAS2 | 0.7500 | 0.0239 | 100% | 62.5% |
| OAS3 | 0.70833 | 0.0479 | 100% | 62.5% |
| RSAD2 | 0.7500 | 0.0440 | 100% | 75% |
| USP18 | 0.77083 | 0.0362 | 100% | 62.5% |
| AZU1 | 0.7083 | 0.0678 | 67% | 100% |
| BPI | 0.7500 | 0.1115 | 67% | 87.5% |
| CEACAM8 | 0.70833 | 0.1332 | 67% | 100% |
| CRISP3 | 0.7500 | 0.1840 | 67% | 87.5% |
| CTSG | 0.70833 | 0.1310 | 67% | 100% |
| DEFA4 | 0.70833 | 0.0584 | 67% | 100% |
| ELANE | 0.66667 | 0.1040 | 67% | 100% |
| LCN2 | 0.70833 | 0.1247 | 50% | 100% |
| LTF | 0.70833 | 0.1309 | 67% | 87.5% |
| MMP8 | 0.70833 | 0.0908 | 50% | 100% |
| MPO | 0.62500 | 0.2466 | 67% | 87.5% |
| RNASE2 | 0.79167 | 0.0243 | 67% | 100% |
| RNASE3 | 0.91667 | 0.0012 | 100% | 75% |

### REFERENCES

1. Fransen J, van Riel P L (2005) The Disease Activity Score and the EULAR response criteria. Clin Exp Rheumatol 23: S93-99.
2. Trapnell C, Roberts A, Goff L, Pertea G, Kim D, et al. (2012) Differential gene and transcript expression analysis of RNA-seq experiments with TopHat and Cufflinks. Nat Protoc 7: 562-578.
3. Anders S HTSeq: Analysing high-throughput sequencing data with Python. http://www-huber.embl.de/users/anders/HTSeq/doc/overview.html
4. Mantovani A, Cassatella M A, Costantini C, Jaillon S (2011) Neutrophils in the activation and regulation of innate and adaptive immunity. Nat Rev Immunol 11: 519-531.
5. Wright H L, Thomas H B, Moots R J, Edwards S W (2013) RNA-Seq Reveals Activation of Both Common and Cytokine-Specific Pathways following Neutrophil Priming. PLoS One 8: e58598.
6. Ramskold D, Wang E T, Burge C B, Sandberg R (2009) An abundance of ubiquitously expressed genes revealed by tissue transcriptome sequence data. PLoS Comput Biol 5: e1000598.
7. Rowley μW, Oler A J, Tolley N D, Hunter B N, Low E N, et al. (2011) Genome-wide RNA-seq analysis of human and mouse platelet transcriptomes. Blood 118: e101-111.
8. Zhang X, Ding L, Sandford A J (2005) Selection of reference genes for gene expression studies in human neutrophils by real-time PCR. BMC Mol Biol 6: 4.
9. Robinson M D, McCarthy D J, Smyth G K (2010) edgeR: a Bioconductor package for differential expression analysis of digital gene expression data. Bioinformatics 26: 139-140.
10. Wright H L. Thomas H B, Moots R J, Edwards S W (2015) Interferon gene expression signature in rheumatoid arthritis neutrophils correlates with a good response to TNFi therapy. Rheumatology (Oxford) 54: 188-193.
11. Villanueva E, Yalavarthi S, Berthier C C, Hodgin J B, Khandpur R, et al. (2011) Netting neutrophils induce endothelial damage, infiltrate tissues, and expose immunostimulatory molecules in systemic lupus erythematosus. J Immunol 187: 538-552.

1. A method, comprising analyzing, prior to commencement of anti-TNF therapy, a sample obtained from a subject having an autoimmune or immune-mediated disorder to detect:
(i) the level of a first target molecule indicative of the expression of a Low Density Granulocyte (LDG) gene; and
(ii) the level of a second target molecule indicative of the expression one or more interferon regulated biomarkers selected from the group consisting of CMPK2, IFI6, RSAD2, USP18, IFI44L, LY6E, OAS1, OAS2, OAS3 and IFIT1B,
wherein an elevated level of the first target molecule compared to a first reference level predicts a non-favorable response of the subject to anti-TNF therapy, and an elevated level of the second target molecule compared to a second reference level, with no substantial elevation in the first target molecule compared to the first reference level, predicts a favorable response of the subject to anti-TNF therapy.

2. The method of claim 1, wherein the LDG gene is selected from the group consisting of: AZU1, BPI, CEACAM8, CRISP3, CTSG, DEFA4, ELANE, LCN2, LTF, MMP8, MPO, RNASE2, and RNASE3.

3. (canceled)

4. The method of claim 1, wherein the first reference level and the second reference level are determined from a sample from a subject without an autoimmune or immune-mediated disorder.

5. The method of claim 1, wherein the method comprises determining the level of a target molecule indicative of the expression of a biomarker selected from the group consisting of RNASE3, and RNASE2.

6. The method of claim 5, wherein an elevated level of RNASE3 comprises at least 0.75, 1, or 1.2 fold or more increase in expression relative to a reference value.

7.-10. (canceled)

11. The method of claim 1, wherein the method comprises determining the levels of CMPK2, IFI44L, IFIT1B and RNASE3.

12. (canceled)

13. The method of claim 1, wherein an autoimmune or immune mediated disorder is selected from the group consisting of Rheumatoid Arthritis, Ankylosing spondylitis, inflammatory bowel disease, vasculitis, juvenile dermatomyositis, scleroderma, Crohn's disease, ulcerative colitis, psoriasis and systemic lupus erythematosus.

14. The method of claim 1, wherein the anti-TNF therapy is selected from the group consisting of proteins, antibodies, antibody fragments, fusion proteins, multivalent binding proteins, small molecule TNF antagonists and similar naturally- or non-naturally-occurring molecules, and/or recombinant and/or engineered forms thereof which inhibit TNF.

15. The method of claim 1, wherein the anti-TNF therapy is selected from the group consisting of a monoclonal antibody, a circulating receptor fusion protein, and a xanthine derivative

16. The method of claim 1, wherein the sample is a whole blood sample.

17. The method of claim 16, wherein the blood sample is a neutrophil fraction, or a purified neutrophil fraction.

18. The method of claim 1 wherein the favorable response to anti-TNF therapy is selected from the group consisting of a reduction in pain, inflammation, swelling, stiffness, an increase in mobility, decreased time to disease progression, increased time of remission.

19.-21. (canceled)

22. The method of claim 1, wherein the target molecules are nucleic acids.

23. (canceled)

24. The method of claim 22, wherein the molecules are detected using a method selected from the group consisting of hybridization techniques, quantitative PCR and high throughput sequencing.

25.-28. (canceled)

29. A kit comprising one or more pairs of primers of Table 4, and one or more of a set of instructions for use, a chart providing reference or baseline values for at least the biomarker corresponding to the primer pairs of the kits, and reagents.

30.-38. (canceled)

39. The method of claim 1, wherein the anti-TNF therapy is selected from the group consisting of etanercept (Enbrel), pentoxifylline and Bupropion.

40. The method of claim 22, wherein the nucleic acid is an mRNA.

41. The method of claim 40, wherein the mRNA is a transcriptome.

42. The method of claim 1, further comprising administering to the subject the anti-TNF therapy when a favorable response of the subject to anti-TNF therapy is predicted; and administering to the subject an alternative therapy when a non-favorable response of the subject to anti-TNF therapy is predicted.

43. The method of claim 42, wherein the alternative therapy is a disease-modifying anti-rheumatic drug (DMARD) selected from a biologic DMARD, methotrexate (MTX), sulfasalazine (SSZ), and hydroxychloroquine (HCQ).

44. The method of claim 42, wherein the anti-TNF therapy is selected from the group consisting of proteins, antibodies, antibody fragments, fusion proteins, multivalent binding proteins, small molecule TNF antagonists, and similar naturally or non-naturally-occurring molecules, and recombinant and/or engineered forms thereof which inhibit TNF.

45. The method of claim 15, wherein the monoclonal antibody is selected from the group consisting of infliximab (Remicade), adalimumab (Humira), certolizumab pegol (Cimzia), and golimumab (Simponi).

46. The method of claim 15, wherein the circulating receptor fusion protein is etarnecept (Enbrel).

47. The method of claim 15, wherein the xanthine derivative is selected from the group consisting of pentoxifylline and Bupropion.

48. A method of treating a subject having an autoimmune or immune-mediated disorder comprising:
analyzing a sample obtained from the subject prior to commencement of the therapy to detect:
(i) the level of a first target molecule indicative of the expression of a Low Density Granulocyte (LDG) gene; and
(ii) the level of a second target molecule indicative of the expression one or more interferon regulated biomarkers selected from the group consisting of CMPK2, IFI6, RSAD2, USP18, IFI44L, LY6E, OAS1, OAS2, OAS3 and IFIT1B,
wherein an elevated level of the first target molecule compared to a first reference level predicts a non-favorable response of the subject to anti-TNF therapy, and an elevated level of the second target molecule compared to a second reference level, with no substantial elevation in the first target molecule compared to the first reference level, predicts a favorable response of the subject to anti-TNF therapy; and
administering to the subject the anti-TNF therapy when a favorable response of the subject to anti-TNF therapy is predicted; or administering to the subject an alternative therapy when a non-favorable response of the subject to anti-TNF therapy is predicted.

49. The method of claim 48, wherein the alternative therapy is a disease-modifying anti-rheumatic drug (DMARD) selected from a biologic DMARD, methotrexate (MTX), sulfasalazine (SSZ), and hydroxychloroquine (HCQ).

50. The method of claim 48, wherein the anti-TNF therapy is selected from the group consisting of proteins, antibodies, antibody fragments, fusion proteins, multivalent binding proteins, small molecule TNF antagonists, and similar naturally or non-naturally-occurring molecules, and recombinant and/or engineered forms thereof which inhibit TNF.


SEQUENCE LISTING (48 sequences)

SEQ ID NO: 1 - DNA, 20 bp, Artificial Sequence
  AZU1-F qPCR primer
cagcagcatgagcgagaatg

SEQ ID NO: 2 - DNA, 20 bp, Artificial Sequence
  AZU1-R qPCR primer
agaggcagtggcagtatcgt

SEQ ID NO: 3 - DNA, 20 bp, Artificial Sequence
  B2M-F qPCR primer
actgaattcacccccactga

SEQ ID NO: 4 - DNA, 20 bp, Artificial Sequence
  B2M-R qPCR primer
cctccatgatgctgcttaca

SEQ ID NO: 5 - DNA, 20 bp, Artificial Sequence
  BPI-F qPCR primer
ggcatgcacacaactggttc

SEQ ID NO: 6 - DNA, 20 bp, Artificial Sequence
  BPI-R qPCR primer
ccagcttgagctctccaaca

SEQ ID NO: 7 - DNA, 20 bp, Artificial Sequence
  CEACAM8-F qPCR primer
gcggaacgtcaccagaaatg

SEQ ID NO: 8 - DNA, 20 bp, Artificial Sequence
  CEACAM8-R qPCR primer
gagtctccggatgtacgctg

SEQ ID NO: 9 - DNA, 20 bp, Artificial Sequence
  CMPK2-F qPCR primer
aggccaacagtgtgtttcgt

SEQ ID NO: 10 - DNA, 20 bp, Artificial Sequence
  CMPK2-R qPCR primer
accgtctgcaggaccttttc

SEQ ID NO: 11 - DNA, 21 bp, Artificial Sequence
  CRISP3-F qPCR primer
tctggaaaccactgcaatgac

SEQ ID NO: 12 - DNA, 21 bp, Artificial Sequence
  CRISP3-R qPCR primer
agcagtaaaagcgggatcctt

SEQ ID NO: 13 - DNA, 20 bp, Artificial Sequence
  CTSG-F qPCR primer
tccgcatcttcggttcctac

SEQ ID NO: 14 - DNA, 20 bp, Artificial Sequence
  CTSG-R qPCR primer
cgtgggccacattgttacac

SEQ ID NO: 15 - DNA, 20 bp, Artificial Sequence
  DEFA4-F qPCR primer
ctgcctcattggtggtgtga

SEQ ID NO: 16 - DNA, 20 bp, Artificial Sequence
  DEFA4-R qPCR primer
ggcgttcccagcatgacatt

SEQ ID NO: 17 - DNA, 20 bp, Artificial Sequence
  ELANE-F qPCR primer
cgtggcgaatgtaaacgtcc

SEQ ID NO: 18 - DNA, 20 bp, Artificial Sequence
  ELANE-R qPCR primer
ttttcgaagatgcgctgcac

SEQ ID NO: 19 - DNA, 20 bp, Artificial Sequence
  IFI44L-F qPCR primer
cctagccatgtgtccttcca

SEQ ID NO: 20 - DNA, 24 bp, Artificial Sequence
  IFI44L-R qPCR primer
gctttcacagctagtaagaggact

SEQ ID NO: 21 - DNA, 20 bp, Artificial Sequence
  IFI6-F qPCR primer
caaggtctagtgacggagcc

SEQ ID NO: 22 - DNA, 20 bp, Artificial Sequence
  IFI6-R qPCR primer
tttcttacctgcctccaccc

SEQ ID NO: 23 - DNA, 20 bp, Artificial Sequence
  IFIT1B-F qPCR primer
tactgggtacgcaatcaccg

SEQ ID NO: 24 - DNA, 20 bp, Artificial Sequence
  IFIT1B-R qPCR primer
gctcgttttaggacgtgcag

SEQ ID NO: 25 - DNA, 20 bp, Artificial Sequence
  LCN2-F qPCR primer
caggactccacctcagacct

SEQ ID NO: 26 - DNA, 20 bp, Artificial Sequence
  LCN2-R qPCR primer
ctgccaggcctaccacatac

SEQ ID NO: 27 - DNA, 20 bp, Artificial Sequence
  LTF-F qPCR primer
ttttcggagcctggatcctc

SEQ ID NO: 28 - DNA, 20 bp, Artificial Sequence
  LTF-R qPCR primer
cgcccctttattcagggctt

SEQ ID NO: 29 - DNA, 20 bp, Artificial Sequence
  LY6E-F qPCR primer
aggacaggctgctttggttt

SEQ ID NO: 30 - DNA, 20 bp, Artificial Sequence
  LY6E-R qPCR primer
agcagcactggcaagaagat

SEQ ID NO: 31 - DNA, 21 bp, Artificial Sequence
  MMP8-F qPCR primer
ccttgctaaggactactgggc

SEQ ID NO: 32 - DNA, 20 bp, Artificial Sequence
  MMP8-R qPCR primer
ctggcccatttgggtttgga

SEQ ID NO: 33 - DNA, 20 bp, Artificial Sequence
  MPO-F qPCR primer
atcgccaacgtcttcaccaa

SEQ ID NO: 34 - DNA, 20 bp, Artificial Sequence
  MPO-R qPCR primer
catgggctggtaccgattgt

SEQ ID NO: 35 - DNA, 21 bp, Artificial Sequence
  OAS1-F qPCR primer
gattctgctggctgaaagcaa

SEQ ID NO: 36 - DNA, 20 bp, Artificial Sequence
  OAS1-R qPCR primer
gagtgtgctgggtctatgag

SEQ ID NO: 37 - DNA, 23 bp, Artificial Sequence
  OAS2-F qPCR primer
ctcctcctttttccttccagtct

SEQ ID NO: 38 - DNA, 20 bp, Artificial Sequence
  OAS2-R qPCR primer
aagcaccgagagcaagatca

SEQ ID NO: 39 - DNA, 21 bp, Artificial Sequence
  OAS3-F qPCR primer
tggaccatcaactacaacgcc

SEQ ID NO: 40 - DNA, 23 bp, Artificial Sequence
  OAS3-R qPCR primer
atccaggatgataggcctgaacc

SEQ ID NO: 41 - DNA, 20 bp, Artificial Sequence
  RNASE2-F qPCR primer
gtggaagccaggtgccttta

SEQ ID NO: 42 - DNA, 20 bp, Artificial Sequence
  RNASE2-R qPCR primer
catgtttgctggtgtctgcg

SEQ ID NO: 43 - DNA, 20 bp, Artificial Sequence
  RNASE3-F qPCR primer
gcacgtatgcagacagacca

SEQ ID NO: 44 - DNA, 20 bp, Artificial Sequence
  RNASE3-R qPCR primer
ggtgaactggaaccacagga

SEQ ID NO: 45 - DNA, 20 bp, Artificial Sequence
  RSAD2-F qPCR primer
tgctgggaagctcttgagtg

SEQ ID NO: 46 - DNA, 20 bp, Artificial Sequence
  RSAD2-R qPCR primer
agctagcagccagaaggttg

SEQ ID NO: 47 - DNA, 20 bp, Artificial Sequence
  USP18-F qPCR primer
agtccccggcagatcttgaa

SEQ ID NO: 48 - DNA, 20 bp, Artificial Sequence
  USP18-R qPCR primer
aaaccaaccaggccatgagg